(19) United States
(12) Patent Application Publication
(10) Pub. No.: US 2019/0195897 A1
(43) Pub. Date: Jun. 27, 2019

(54) GLYCOSYLATED METABOLITES

(71) Applicant: UNIVERSITEIT LEIDEN, Leiden (NL)

(72) Inventors: Johannes Maria Franciscus Gerardus AERTS, Leiden (NL); Hermen Steven OVERKLEEFT, Leiden (NL)

(73) Assignee: UNIVERSITEIT LEIDEN, Leiden (NL)

(21) Appl. No.: 15/739,043

(22) PCT Filed: Jun. 24, 2016
(86) PCT No.: PCT/NL2016/050450

§ 371 Date: Dec. 21, 2017

(30) Foreign Application Priority Data
Jun. 24, 2015 (NL) 15173553.7

(51) Int. Cl. G01N 33/82; A61K 31/445; G01N 33/68; G01N 33/92

(52) U.S. Cl. CPC G01N 33/82; A61K 31/445; G01N 33/6893; G01N 2560/00; G01N 2440/38; G01N 2400/00; G01N 33/92

(57) ABSTRACT

The invention provides means and methods for detecting a glycosylated metabolite in a sample comprising adding to the sample a compound comprising a labelled glycosyl group coupled via an O-glycosidic bond to an aglycon with a specific structural formula wherein the glycosyl group comprises at least one isotope and/or a side chain being a label for detection, and wherein the sample is screened for the presence of a glycosylated metabolite with the glycosyl group other than a glucosylceramide. The invention further provides a method of treating a disorder caused by accumulation of a glycosylated metabolite other than glucosylceramide in a subject comprising administering to the subject in need thereof a therapeutically effective amount of a glucosylceramide synthase (GCS) inhibitor.

### FIELD OF THE INVENTION

The present invention relates to a method for detecting a glycosylated metabolite in a sample from a subject, and further relates to a method for typing a sample from a subject based on a level of a glycosylated metabolite in the sample. This can be used for typing the sample as a sample from a subject suffering from or with a predisposition for a disorder caused by accumulation of a glycosylated metabolite. The present invention additionally relates to the use of a glucosylceramide synthase (GCS) inhibitor in treatment of a disorder caused by accumulation of a glycosylated metabolite in a subject.

### BACKGROUND OF THE INVENTION

Glycosylation in biology is the enzymatic process for attaching glycans to a substrate such as proteins, lipids, or other organic molecules. Glycosylation is a form of co-translational and post-translational modification. Glycans serve a variety of structural and functional roles in membrane and secreted proteins. The majority of proteins synthesized in the rough ER undergo glycosylation. It is an enzyme-directed site-specific process. Glycosylation occurs in the cytoplasm as well as in the nucleus, and accordingly glycosylated substrates are present in both the cytoplasm and nucleus.

Glycosylation of molecules serves various functions. For instance, glycosylation plays a role in proper folding of proteins. There are various proteins that do not fold correctly unless they are glycosylated. There are also proteins that are not stable unless they contain polysaccharides linked at the amide nitrogen of certain asparagines. Experiments have shown that these proteins in unglycosylated form degrade quickly. Glycosylation also plays a role in cell-cell adhesion via sugar-binding proteins called lectins, which recognize specific carbohydrate moieties. The importance of glycosylation is evident from the more than 40 disorders that have been reported in humans to be associated with dysfunctional glycosylation. No effective treatment is known for any of these disorders.

In addition to proteins, other organic molecules such as lipids are also a subject of glycosylation. Of the disorders known in humans related to glycosylation a substantial part belong to the group of disorders related to glycosylated lipid accumulation. Glycosylated lipids thus form an interesting subject for investigation. The major glucosylated metabolite in humans is the simplest glycosphingolipid named glucosylceramide (ceramide-beta-glucoside: GlcCer). Glucosylation is a type of glycosylation in which the glycan involved is based on the sugar glucose. Another example of a type of glycosylation is xylosylation based on the sugar xylose. In this context the popular term “sugar” and the term “saccharide” are used interchangeably. GlcCer is formed in the cytosol by the action of the enzyme glucosylceramide synthase GCS, encoded by the UGCT gene, that transfers glucose from the donor UDP-glucose to ceramide. GlcCer is ubiquitous in mammalian cells, and particularly located in the cell membrane (1). Its presence in plants and some fungi is also documented. GlcCer is formed by the enzyme glucosylceramide synthase (GCS, EC2.4.1.80). The transferase, firstly cloned by Hirabayashi and colleagues (8), is located at the cytosolic leaflet of Golgi apparatus where it transfers the glucose-moiety from UDP-glucose to ceramide (9). Cleavage of the glucosyl-group of GlcUer can be achieved by the lysosomal enzyme glucocerebrosidase (GBA, E.C.3.2.1.45), external Ids are HGNC: 4177; Entrez Gene: 2629; Ensembl.: ENSG00000177628; OMIM: 606463 and UniProtKB: P04062, which is needed for hydrolysis. This enzyme is well studied since its deficiency underlies Gaucher disease (GD).

Gaucher's disease (GD), the most common of the lysosomal storage diseases (LSD), is a form of sphingolipidosis, as it involves dysfunctional metabolism of sphingolipids, causing sphingolipids to accumulate in cells and certain organs of the patient. The disorder is characterized by bruising, fatigue, anemia, low blood platelets, and enlargement of the liver and spleen. It is caused by a hereditary deficiency of GBA (also known as glucosylceramidase). When the enzyme is defective, GlcCer accumulates, particularly in white blood cells, most often macrophages (mononuclear leukocytes). GlcCer can collect in the spleen, liver, kidneys, lungs, brain, and bone marrow. Manifestations may include enlarged spleen and liver, liver malfunction, skeletal disorders and bone lesions that may be painful, severe neurologic complications, swelling of lymph nodes and (occasionally) adjacent joints, distended abdomen, a brownish tint to the skin, anemia, low blood platelets, and yellow fatty deposits on the white of the eye (sclera). Persons affected most seriously may also be more susceptible to infection. The disease is caused by a recessive mutation in a gene located on chromosome 1 and affects both males and females.

Assisted by the small activator protein saposin C, GBA in lysosomes degrades GlcCer to ceramide and glucose, the penultimate step in glycosphingolipid catabolism (13). Accordingly deficient GBA activity in GD patients results in accumulation of GlcCer in lysosomes, and most prominently in macrophages. The enlarged lipid-laden macrophages are referred to as Gaucher cells and occur in spleen, liver, bone marrow and lung. Their accumulation in various tissues is thought to give rise to various symptoms such as hepatosplenomegaly, pancytopenia and skeletal complications (13). Presence of Gaucher cells in viscera is reflected by increased amounts of protein markers of these cells in the blood of GD patients. Examples of such plasma biomarkers of GD are the chitinase chitotriosidase and chemokine CCL1.8 (14). Symptomatic GD patients also show a marked increase in plasma glucosylsphingosine (GlcSph), the deacylated form of GlcCer (15, 16). The non-neuronopathic (type 1) variant of GD is presently treated by enzyme replacement therapy, implying chronic two-weekly intravenous infusion of recombinant enzyme (17). The therapeutic enzyme is modified in its N-linked glycans to maximally expose terminal mannose residues ensuring delivery to lysosomes of macrophages. An alternative treatment of type 1 GD, named substrate reduction, is based on oral administration of an inhibitor of glucosylceramide synthase (18-20). This substrate reduction therapy aims to restore the balance between formation and degradation of GlcCer in GD patients by reducing the biosynthesis of GlcCer. Successful therapy of type 1 GD patients results in reduction of visceral Gaucher cells, and corrections in circulating glucosylsphingosine and biomarkers of Gaucher cells (13).

For some of the complex pattern of symptoms occurring in patients suffering from Gaucher's disease there is thus far no explanation. For example, at least some GD patients show osteoporosis and unexplained abnormal vitamin D metabolism. Moreover, at least some GD patients as well as carriers of the disease are also at significantly increased risk for developing alpha-synucleinopathies like Parkinsonism and Lewy-Body dementia, again for unknown reasons. Thus there is a need to understand what causes these unexplained symptoms to occur in patients with a dysfunctional metabolism of sphingolipids and to investigate possible ways of treatment of these conditions.

Besides the glycosphingolipid GlcCer, there are other lipids with glucosylated structures reported in membranes of higher eukaryotic cells, including glycerolipid.s, sterols and other sphingolipids. For instance, glucosyldiacylglycerol (G1cDG) has been identified in various plants, but its presence in mammalian cells is comparatively poorly documented (2, 3). Likewise, sterol-glucosides are known to occur in plants (4), but again their existence in mammalian cells is little studied so far. Indications for the existence of glucosyl-β-D-cholesterol or 1-O-cholesteryl-β-D-glucopyranoside (GlcChol) in mammalian cells were first provided by Murofushi and co-workers. They described its occurrence in cultured human fibroblasts and gastric mucosa (5, 6). Heat shock was found to increase biosynthesis of GlcChol and subsequently induce HSP70 (7). In a recent study, Akiyma and colleagues showed that GM-95 cells deficient in GCS are unable to synthesize GlcChol without the addition of exogenous GlcCer (10). The same researchers demonstrated in addition that, at least in vitro, GBA generates through transglucosylation 25-NBD-cholesterol-glucoside from GlcCer and artificial 25-NBD-cholesterol GlcChol (11). Such ability of GBA to perform transglucosylation was earlier demonstrated by Glew and co-workers showing catalyzed transfer of the glucose moiety from 4-methylumbelliferyl-β-glucoside to retinol and other alcohols (12).

Presently there is hardly any documentation on the possible role of transglucosylation in disorders caused by a dysfunctional metabolism of sphingolipids. In view of the unexplained symptoms occurring in patients with a sphingolipidosis, particularly GD, and a possible role of glycosylated lipids other than GlcCer therein, there is a particular need for further investigation of the transglucosylation process and the resulting glycosylated lipids other than GlcCer, particularly in cells of subjects suffering from a disorder related to a dysfunctional metabolism of sphingolipids. A better understanding of the factors involved in transglucosylation between lipids, particularly in vivo, may contribute to gain new insights in the mechanisms underlying lysosomal storage diseases, particularly sphingolipidosis, in particular GD, and may be used to differentiate a healthy individual from an individual suffering from or susceptible for such a lysosomal storage disease.

Accordingly the present invention thus aims among others to provide means and methods of detecting a glycosylated metabolite other than GlcCer in a sample, as well as means and methods for treatment of a disorder caused by accumulation of a glycosylated metabolite in a subject.

### SUMMARY OF THE INVENTION

The present invention provides a method of detecting a glycosylated metabolite in a sample comprising adding to the sample a compound comprising a labelled glycosyl group coupled via an O-glycosidic bond to an aglycon and of the following structural formula:

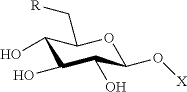

in which
R=a hydroxyl or a detection label; and
X=an aglycon;

wherein the glycosyl group comprises at least one isotope and/or an R side chain being a label for detection, and wherein the sample is screened for the presence of a glycosylated metabolite with the glycosyl group other than a glucosylceramide. The method preferably comprising incubating the sample subsequent to addition of the compound under conditions that allow a transglycosylation reaction to occur using the compound as a substrate for the transglycoslation. The screening preferably involves the detection of a molecule comprising the labelled glycosyl group which is not said compound.

For identification of new products of a trans-glycosylation reaction the labelled compound is a suitable means as donor of a glycosyl group to a possible acceptor of the glycosyl group present in the sample, which acceptor after transfer of the labelled glycosylgroup from the donor thereon, for instance after transglycosylation by means of a glycosyltransferase, is detectable. The label may be a suitable R side chain, such as for instance a fluorescent label such as a boron-clipyrromethene (BODIPY), a NBD fluorophore, or a cyanine dye such as Cy5 for visual inspection of the presence of a glycosylated metabolite in the sample or for detection using a separation technique such as High Performance Thin-layer chromatography (HPTLC) and fluorescence scanning. To enable convenient labelling of the glycosyl group the R-group of the compound in a particular embodiment of the method according to the invention is an azide (N₃), which allows for “click reaction” chemistry to replace the label as desired. Particularly the compound in the method according to the present invention in this case is a C6-azide-Glc-X, preferably C6-azide-GlcCer. In this embodiment the sample is preferably incubated to allow a trans-glycosylation reaction to occur.

To enable quantitative detection of the natural occurrence of GlcX in samples of a subject a sensitive assay for quantification of GlcX in plasma, cells and tissues is needed. Liquid chromatography mass spectrometry (LC-MS) is a well-known technique that has very high sensitivity and selectivity for general detection and potential identification of chemicals of particular masses in complex mixtures, and is a suitable means for detecting the occurrence of GlcX, such as GlcChol, in samples of a subject. In order to not require correction for the sample matrix (ion suppression or ion enhancement), particularly an isotope labelled GlcX may be used as internal standard, since the extraction efficiency, chromatographic behavior and ionization characteristics of the natural compound and the corresponding isotope labeled compound are identical.

Accordingly, in a further particular embodiment of the method according to the invention the glycosyl group of the compound comprises at least one ¹³C-isotope for detection using LC-MS/MS. A preferred labelled compound in the method according to the invention is a ¹³C₆Glc-X compound, more particularly ¹³C₆GlcCer or ¹³C₆GlcChol and specifically ¹³C₆-β-GlcChol. The present invention also relates to ¹³C₆GlcCer or ¹³C₆GlcChol, particularly ¹³C₆-⊖-GlcChol, as a compound and uses thereof other than in a method according to the present invention. The enable quantitative detection of a GlcX compound in a sample the invention further provides a method for detecting a glycoside in a sample comprising adding a radio-isotope labeled form of said glycoside to the sample as an internal standard, the method further comprising detecting total and radio-isotope labeled glycoside in the sample and quantitating the glycoside in the sample. The radio-isotope is preferably present in the saccharide of said glycoside. The radio-isotope is preferably ¹³C. The radio-isotope labeled glycoside is preferably a ¹³C-glycoside. The radio-labeled glycosylated metabolite is preferably a ¹³C6-glycoside. The glycoside preferably comprises a saccharide linked to an aglycone. The aglycone is preferably a sterol, a monoacylglycerol (endocannabinoid), a (diacyl)glycerol, a retinol, a dihydrocalciferol, a tocopherol, a geraniol, a farnesol, a serine, a threonine, or a tryptophan. The aglycone is preferably not ceramide.

As used herein the term “glycosylated metabolite” is preferably a glycoside. In a preferred embodiment the term “glycosylated metabolite” is replaced by the term “glycoside”. A glycoside is preferably a saccharide linked to an aglycone.

In a particular embodiment of the method according to the invention the glycosylated metabolite is a glucosylated or xyl.osylated metabolite, in particular a glucosylated or xylosylated sterol, monoacylglycerol (endocannabinoid), (diacyl)glycerol, retinol, dihydrocalciferol, tocopherol, geraniol, farnesol, serine, threonine, or tryptophan. Such metabolites are interesting substrates of which the natural occurrence of the glycosylated form may result in a change in physico-chemical properties, which may be of physiological relevance.

In a preferred embodiment of the method according to the invention the aglycon X is selected from a group comprising 4-methylumbelliferone, p-nitrophenol, dopamin, serotonin, vitamin D precursor, calciferol or cholecalciferol, dihydrocalciferol, monoacylglycerol (endocannabinoid), (diacyl)glycerol, retinol, tocopherol, geraniol, farnesol, serine, threonine, tryptophan, and sterol, in particular ceramide or cholesterol. In a particularly preferred embodiment of the method according to the invention the aglycon X is selected from a group comprising 4-methylumbelliferone, p-nitrophenol, dopamin, serotonin, vitamin D precursor, calciferol or cholecalciferol, dihydrocalciferol, monoacylglycerol (endocannabinoid), (diacyl)glycerol, retinol, tocopherol, geraniol, farnesol, serine, threonine and tryptophan. This group of synthetic or naturally occurring aglycons are found suitable substrates for the compound to function as a glycosyl-group donor in a transglycosylation process.

In a further preferred embodiment of the method according to the invention the sample comprises a glycosyltransferase, in particular a glucocerebrosidase (GBA or GBA1) or a glucosylceramidase (GBA2). The glycosyltransferase enzyme allows for the transfer of the labelled glycosyl-group from the used compound to a glycosyl-group acceptor present in the sample. Accordingly the glycosyltransferase promotes the labelling of the glycosylated metabolite to be detected. In a particular embodiment the method according to the invention comprises that the glycosyltransferase is added to the sample. A glycosyltransferase, for example recombinant GBA, may be added to the sample for instance in the event the sample does not comprise a functional glycosyltransferase of its own, or in the event it is not known or not certain that the sample comprises a glycosyltransferase of its own.

The sample in the method of the invention preferably is a sample from an animal subject, particularly a mammal subject, more particularly a human subject. A further preferred embodiment of the method according to the invention comprises that the sample is a sample from a subject, particularly a human subject, suffering from a disorder caused by accumulation of excessive amounts of a glycosylated metabolite, and in a particularly preferred embodiment the disorder is one or more of lysosomal glycosphingolipid storage disorder, osteoporosis, abnormal vitamin D metabolism or an alpha-synucleinopathy (Parkinson's, Lew-Body dementia).

Results of quantitative detection of a glycosylated metabolite other than GlcCer in samples of healthy subjects as compared to subjects suffering from a lysosomal glycosphingolipid storage disorder, indicate that there is a correlation between relatively high levels of glycosylated metabolite present in a sample and a risk for the subject of that sample for suffering from a lysosomal storage disorder. Accordingly, the present invention further relates to a method for typing a sample from a subject based on a level of a glycosylated metabolite in the sample for typing the sample as a sample from a subject suffering from or with a predisposition for a disorder caused by accumulation of the glycosylated metabolite. In particular the method of typing a sample from an individual according to the present invention comprises determining a level of at least one glycosylated metabolite other than glucosylceramide in a relevant sample from the individual, comparing said level with a reference; and typing said sample as a sample from an individual with a predisposition for one or more of the disorders lysosomal glycosphingolipid storage disorder, osteoporosis, abnormal vitamin D metabolism or an alpha-synueleinopathy on the basis of said comparison.

In a particular embodiment of the method of typing a sample from an individual according to the present invention the at least one glycosylated metabolite is one or more of the group comprising sterol, (diacyl)glycerol, retinol, tocopherol, geraniol, farnesol, serine, threonine, or tryptophan.

The present invention moreover relates to a glucosylceramide synthase (GCS) inhibitor for use in treatment of a disorder caused by accumulation of a glycosylated metabolite other than glucosylceramide in a subject. Particularly the invention relates to a glucosylceramide synthase (GCS) inhibitor for use in treatment of a disorder caused by accumulation of a glycosylated metabolite other than glucosylceramide in a subject, said disorder not comprising Gaucher Disease (GD). The present invention also relates to a method of treating a disorder caused by accumulation of a glycosylated metabolite other than glucosylceramide in a subject comprising administering to the subject in need thereof a therapeutically effective amount of a glucosylceramide synthase (GCS) inhibitor. Particularly the invention relates to a method of treating a disorder caused by accumulation of a glycosylated metabolite other than glucosylceramide in a subject comprising administering to the subject in need thereof a therapeutically effective amount of a glucosylceramide synthase (GCS) inhibitor, wherein the disorder does not comprise Gaucher Disease.

In a preferred embodiment the glucosylceramide synthase (GCS) inhibitor for use or in the method according to the present invention is selected from the group comprising miglitol, miglustat, eliglustat or a biphenyl-substituted deoxynojirimycin derivative.

Particularly the biphenyl-substituted deoxynojirimycin derivative as glucosylceramide synthase (GCS) inhibitor for use according to the present invention or in a method according to the present invention is a biphenyl-substituted D-gluco-deoxynojirimycin or a biphenyl-substituted L-ido-deoxynojirimycin, and more in particular a biphenyl-substituted L-ido-deoxynojirimycin selected from the group with the following structural formulas:

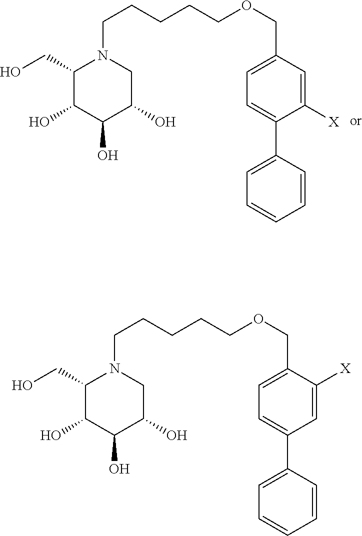

wherein X is F or CF₃.

N-alkylated deoxynojirimycin derivatives are shown to be dual glucosylceramide synthase/neutral glucosylceramidase inhibitors, which render these compounds particularly suitable for the treatment of neuropathological lysosomal storage disorders. Biphenyl-substituted L-ido configured deoxynojirimycin derivatives are selective for glucosylceramidase and the nonlysosomal glucosylceramidase, while demonstrating no intestinal glycosidase inhibitory capacity. Specifically the biphenyl moieties are less prone to the formation of toxic metabolites, as compared to other possible moieties such as naphthyl- and pyrenyl moieties, have good drug-like properties and are attractive from a medicinal chemistry point of view to make various structural modifications. The chemical modifications at X provide different selectivity and potency profiles, as well as improved drug metabolism and pharmacokinetics (DMPK) properties. These and various other inhibitors of GCS are for instance described in Amar T. Ghisaidoobe, Johannes M. F. G. Aerts, Herman S. Overkleeft et al., Journal of Medicinal Chemistry. 2014 57:9096-9104. “Identification and Development of Biphenyl Substituted Iminosugars as Improved Dual Glucosylceramide Synthase/Neutral Glucosylceramidase Inhibitors” which is incorporated by reference herein in its entirety.

The glycosylated metabolite of which the accumulation causes the disorder for which the glucosylceramide synthase (GCS) inhibitor is used as treatment according to the present invention is a dopamine, serotonin, vitamin D precursor, calciferol, cholecalciferol, dihydrocalciferol, monoacylglycerol (endocannabinoid), (diacyl)glycerol, retinol, tocopherol, geraniol, farnesol, serine, threonine, tryptophan or sterol, and in particular cholesterol. Particularly the glucosylceramide synthase (GCS) inhibitor for use or in the method according to the present invention is used to treat one or more of osteoporosis, abnormal vitamin D metabolism or an alpha-synucleinopathy (Parkinson's, Lew-Body dementia).

### DESCRIPTION OF THE DRAWINGS

These and other aspects of the present invention are further elucidated by the appended drawings, which form part of the present application. The drawings are not in any way meant to reflect a limitation of the scope of the invention, unless this is clearly and explicitly indicated.

FIG. 1 illustrates:

FIG. 1A: MS-scan of pure GlcChol-Glc and its isotope. The ammonium adduct is the most abundant M/Z for both compounds. The product ion M/Z 369.4 is the common fragment for both compounds. Shown are the parent scans of product ion 396.4 of GlcChol-Glc and

¹³C6 -labelled Glc-Chol, [M+NH⁴]⁺, 566.6 for GlcChol and 572.6 for ¹³C6 GlcChol. The [M+H]⁺ and [M+Na]⁺ are the minor M/Zs. M/z 571.6 represents the sodium adduct of GlcChol.

FIG. 1B: The structure of Chol-Glc and its isotope ¹³C6-labelled Glc-Chol, their fragmentation pattern M/Z 369.4 is the product ion of both compounds after loss of glucose moiety.

FIG. 1C: Elution pattern of Chol-Glc (m/z 566.6>369.4) and ¹³C6 -labelled Chol-Glc (m/z 572.6>369.4) from UPLC.

FIG. 1D: Linearity of GlcChol quantification and its complete digestion with rGBAl. (5 IU) for 60 min at 37 C.

FIG. 2 illustrates GlcChol in liver and thymus of wild type, GBA-deficient and GBA2-deficient mice.

FIG. 2A: GlcChol (nmol/g wet weight) in various tissues of wild type mice.

FIG. 2B: GlcChol (nmol/g wet weight) in thymus of wild type, GBA2^{-/-} and LIMP2^{-/-} mice.

FIG. 2C: GlcChol (nmol/g wet weight) in liver of wild type, GBA2^{-/-} and LIMP2^{-/-} mice.

FIG. 2D: GlcChol (pmol/ml) in plasma of wild type, GBA2^{-/-} and LIMP2^{-/-} mice.

FIG. 2E: Plasma GlcChol (nM) in normal, type 1 GD mice untreated and treated with GENZ 112638.

FIG. 2F: Liver GlcChol in wt mico, type 1 GD induced mice untreated, type 1 GD treated with lentiviral GBA cDNA gene therapy with macrophage specific promotor (CD68), general strong promotor (PGK) or non-functional SFFV promotor.

FIG. 3 illustrates in vitro transglucosylation of 25-NBD CholGlc by GBA and GBA2.

FIG. 3A: Recombinant rGBA1 and lysates of cells with overexpression of GBA2 and GBA3 were incubated for 0 and 1 hour with 25-NBD-cholesterol in the presence of indicated GlcCer as donor. Formation of 25-NBD-cholesterol glucoside was detected by HPTLC and fluorescence scanning.

FIG. 3B: Recombinant rGBA1 and lysates of cells with overexpression of GBA2 and GBA3 were incubated for 0 and 1 hour with GlcChol in the presence of NBD-Cer. Formation of NBD-GlcCer was detected by HPTLC and fluorescence scanning.

FIG. 4 illustrates GlcChol in CO7 cells manipulated in GSL metabolizing enzymes.

FIG. 4A: GlcChol (nmol/g protein) in COS cells without overexpression of enzymes (control), overexpressed GBA2, GCS and both (GBA2+GCS). Cells were incubated for 2 days with indicated inhibitors of GBA2 (AMP-DNM) and GBA (CBE).

FIG. 4B: GlcChol (umol/g protein) in same cells.

FIG. 5 illustrates GlcChol abnormalities in NYC.

FIG. 5A: GlcChol (nmol/g wet weight) in liver of Npc−/− mice

FIG. 5B: GlcChol (nmol/g wet weight) in liver of spm−/spm NPC mice

FIG. 5C: GlcChol (pmol/mg protein) in RAW 267 cells incubated with indicated concentration U18666A for 1 day in absence and presence of conduri.tol B-epoxide (CBE) inhibiting GBA.

FIG. 5D: GlcChol (pmol/mg protein) in RAW 267 cells incubated with 10 uM U18666A for 1 day and in subsequent absence and presence of 1 mM β-methylcyclodextrin (β-mCD) reducing intralysosomal cholesterol.

FIG. 6 illustrates plasma GlcChol in LSD patients and normal individuals.

FIG. 6A: Plasma GlcChol in type 1 GD patients, NPC patients NPC carriers and normal individuals

FIG. 6B: Plasma GlcChol/Chol in type 1 GD patients, NPC patients, NPC carriers and normal individuals.

FIG. 6C: Reduction in plasma GlcChol following Eliglustat treatment.

### DETAILED DESCRIPTION OF THE INVENTION

Unless otherwise defined, all technical and scientific terms used herein have the same meaning as commonly understood by one of ordinary skill in the art to which this invention belongs. Methods and materials are described herein for use in the present invention. However other suitable methods and materials known in the art can also be used. The materials, methods, and examples are illustrative only and not intended to be limiting, unless so indicated. All publications and other references mentioned herein are incorporated by reference in their entirety. In case of conflict, the present specification, including definitions, will control.

Quantification of GlcChol by LC-MS/MS.

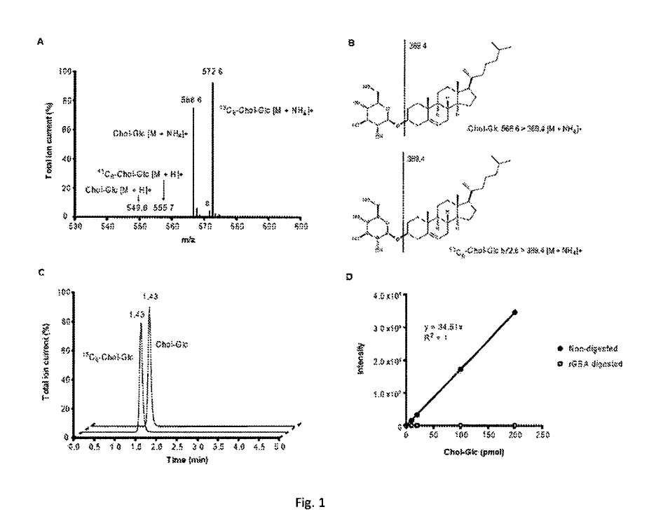

The physiological significance of glucosylation of cholesterol is hypothetically great since it renders the molecule far more water soluble. To establish whether GlcChol physiologically occurs in mammals, firstly a LC-MS/MS procedure was developed for its quantitative detection. For this purpose, a ¹³C-isotope labeled cholesterolglucoside was synthesized to be used as internal standard in sensitive quantitative detection of GlcChol by LC-MS/MS. The use of the isotope labeled compound as internal standard avoids the need for corrections for extraction efficiency, chromatographic behavior and ionization efficiency, during quantification GlcChol. To prevent undesired adduct formation, lipids were extracted in the absence of additional salts. To stimulate formation of desired ammonium adduct we incorporated 10 mM ammonium in the eluent. Sensitive quantitative measurement of GlcChol proved feasible with ¹³C-isotope labeled GlcChol as internal standard (FIG. 1A-C). The limit of detection (LOD) was 0.5 pmol/mL plasma, with a signal to noise ratio of 3 and a limit of quantitation (LOQ) of 0.9 pmol/mL plasma with a signal to noise ratio of 10. GlcChol was found to be an excellent substrate for recombinant GBA1, even at sub-optimal conditions (absence of Triton-X-100 and sodium taurocholate) (FIG. 1D).

Demonstration of natural occurrence of GlcChol in mice.

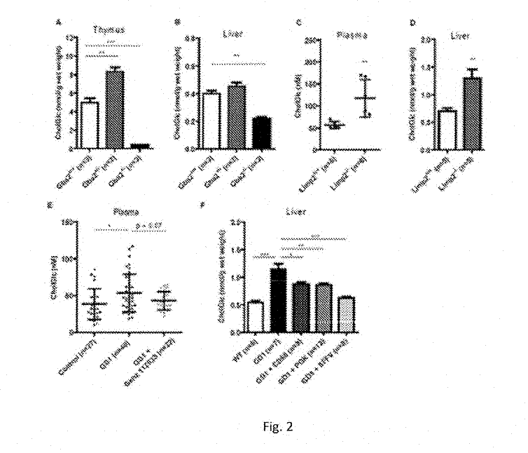

Various tissues of wild type mice were examined for presence of GlcChol. Relatively high amounts of glucosylated sterol were noted for thymus (FIG. 2). The identity of the quantified structure (m/z 566.6) in the tissues was confirmed by its digestion by recombinant GBA1 (rGBA1, Cerezyme®), showing that in wild type animals more than 90% of the lipid measured is indeed Chol-beta-D-Glc.

The concentration of GlcChol in liver and thymus was next determined for tissues collected from normal mice, animals lacking GBA2 and LIMP-2 KO mice with markedly reduced GBA due to impaired transport to lysosomes (39, 40). As shown in FIG. 2A and FIG. 2B, the GlcChol concentration was markedly lower in tissues of GBA2-deficient animals, especially in thymus. In contrast, in the GBA-deficient LIMP-2 KO mice no reduction in GlcChol, but rather a small increase in levels was observed (FIG. 2A, 2B). An increase in GlcChol was also observed in liver of mice with an induced GBA deficiency in the white blood cell lineage (FIG. 2C). Efficient correction of GBA deficiency by gene therapy led to a concomitant reduction of GlcChol (FIG. 2C). Treatment of mice with induced Gaucher disease with GCS inhibitor GENZ 112638 led to partial reduction in plasma GlcCer and a minor, not statistically significant, reduction of elevated plasma GlcChol (FIG. 2D).

The relative high amount of the glucosylated sterol in the thymus, several nanomoles per gram wet weight, is striking. This observation deserves special attention in view of noted abnormalities in NKT cells and B-cells in GBA-deficient GD patients (44). It has been speculated by Mistry and colleagues that elevated GlcCer or GlcSph via binding to CD1 may be causing this (44). It will be now of interest to study whether GlcChol interacts with CD1 since abnormalities in concentration of this lipid in GBA-deficient GD patients are likely. Indeed, GBA is able to form GlcChol by transglucosylation of cholesterol, at least in vitro. Artificial β-glucosides like 4-methylumbelliferyl-β-glucoside may in vitro act as donor in this reaction as well as natural GlcCer. GlcChol is on the other hand also an excellent substrate for hydrolysis by GBA. These indications suggest the importance of β-glucosidases in GleChol metabolism in vivo.

In vitro transglycosylation by β-glucosidases.

Following the demonstration of the physiological occurrence of GlcChol, the role of various 6-glucosidase in its formation and degradation was investigated. Mammalian cells and tissues contain besides GBA other β-glucosidases which are capable of degrading GlcCer. All cells express the membrane-associated non-lysosomal glucosylceramidase, named GBA2 (21-23), KIAA 1605, external Ids HGNC: 18986; Entrez Gene: 57704; Ensembl: ENSG00000070610; OMIM: 609471 and UniProtKB: Q9HCG7. This enzyme is not deficient in GD patients. In fact, a compensatory overexpression of GBA2 in materials of GD has been reported (24). GBA2, claimed to be located at the endoplasmic reticulum in hepatocytes (22) and at the endo-lysosomal system in other cell types (23), degrades GlcCer without need for an activator protein as GBA, and it differs further from GBA in noted artificial substrate and inhibitor specificity. Finally, some tissues express the enzyme GBA3, also referred to as broad-specific cytosolic β-glucosidase (25). This enzyme shows in vitro a relative poor hydrolytic activity towards GlcCer and is thought to be primarily involved in de-toxification of glucosylated xenobiotics (20). All three human β-glucosidases employ the double displacement mechanism in catalysis and are retaining by virtue. There are many documented examples of transglucosylation mediated by retaining glycosidases (26). Therefore, next to GBA theoretically also GBA2 and GBA3 might be involved in generating GlcChol in vivo.

In these investigations use is made of materials deficient in GBA and GBA2 individuals and mice as well as Niemann-Pick type C, a condition characterized by intralysosomal accumulation of cholesterol. The impact of GD therapies on GlcChol was determined. In addition, the GlcChol metabolism in cells was studied using agents interfering at various steps in glycosphingolipid metabolism by their inhibition of specific enzymes. Specific inhibitors for GCS (27) and GBA2 (28) are available whilst the contribution of GBA in GlcChol metabolism can be distinguished from other β-glucosidases by its exclusive inactivation by β-glucopyranosyl cyclophellitolepoxides (29). These compounds covalently bind to the catalytic nucleophile residue, E340, of GBA. Installment of the fluorophore to the activity-based probe (ABP) makes the cyclophellitol-epoxide sufficiently amphihilic to allow diffusion across membranes and in situ inactivation, and associated fluorescent labeling, of GBA (29, 30).

Both GBA and GBA2, but not GBA3, are able to degrade by hydrolysis as well as synthesize GlcChol at conditions optimal for degradation of 4MU-8-glucoside (FIG. 1D; supplemental Table 1). The importance of substrate and acceptor concentrations regarding the action of GBA and GBA2 in GlcChol metabolism is experimentally demonstrated. This ability of the several GBA's to hydrolyze as well as synthesize GlcChol was studied in vitro using 25-NBD-cholesterol as acceptor and detection of 25-NBD-glucoside formation by TLC and fluorescence scanning. As source of enzyme rGBA1 was used and GBA2 and GBA3 were individually overexpressed in COST-cells. Overexpression of enzymes was checked by measuring enzymatic activity with appropriate assays and visualization of enzymes with the broad-specificity β-glucopyranosyl cyclophellitolaziridine-type ABP (38). rGBA1 and lysates of GBA2 and GBA3 overexpressing cells were incubated with natural glucosylceramide (C16:0-GloCer or C18:1-GlcCer) as donors and 25-NBD-cholesterol as acceptor. Following incubation at optimal conditions for each enzyme (see Materials and Methods section hereinafter), lipids were extracted and subjected to HPTLC. As shown in FIG. 3A, rGBA1 and cell lysates with overexpressed GBA2 generated an additional fluorescent lipid coinciding with 25-NBD-cholesterol-glucoside. This was hardly observed for cell lysates with overexpressed GBA3.

It was observed that incubation of cell lysates with overexpressed GCS with UDP-glucose did not result in formation of GlcChol, whilst concomitantly the same enzyme preparation generated C6-NBD-GleCer from C6-NBD-ceramide (not shown). Thus, in vitro formation of GlcChol by GBA is demonstrated, but not significant formation by GBA3 or GCS. Importantly, considerable in vitro transglycosylase was detected for GBA2 at the conditions used.

It was next studied whether natural GlcChol (40 μM) can also act as donor in transglucosylation by incubating rGBA1 and lysates of cells overexpressing GBA2 or GBA3 in the presence of NBD-ceramide (40 μM) as acceptor. Lysates of cells overexpressing GBA2 and rGBA1 showed prominent formation of fluorescent NBD-GlcCer (FIG. 3B). This was not observed for lysates with overexpressed GBA3 (FIG. 3B). As expected, the findings presented in FIG. 3 illustrate that transglucosylation by both GBA and GBA2 occurs as an equilibrium reaction in which the glucose moiety is reversibly exchanged between cholesterol and ceramide.

Metabolism of GlcChol in cultured COS7 cells.

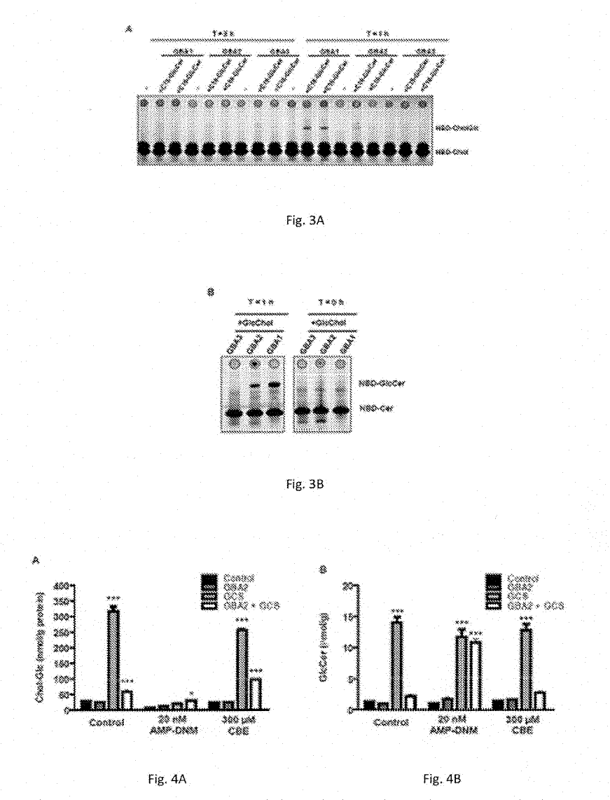

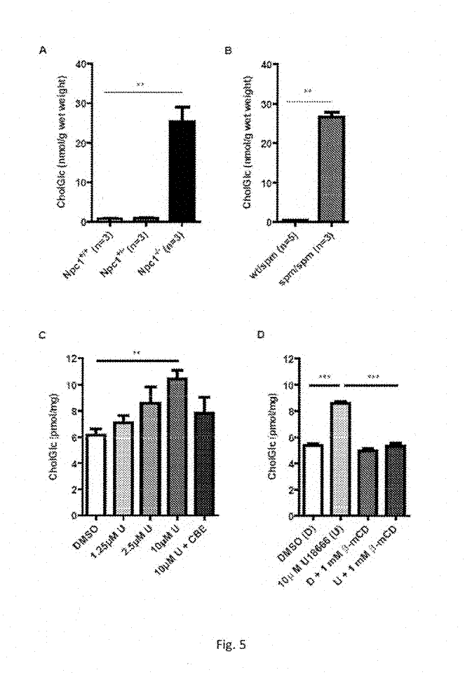

The GlcChol content of cultured COS7 cells and factors influencing this was determined. First the impact of overexpressed GBA2 and GCS was studied. FIG. 4 shows the effect on cellular GlcChol and GlcCer levels. Only overexpression of GCS led to increased levels of GlcCer (FIG. 4B). GlcChol was not changed by overexpression of GBA2, but overexpression of GCS caused a twelve-fold increase. Importantly, inhibition of GBA2 activity with low nanomolar AMP-DNM (28) resulted in reduced cellular GlcChol. Even in cells with overexpressed GCS the elevation in GlcChol was prevented (FIG. 4A). In contrast, inhibition of GBA with CBE did hardly diminish increased cellular GlcChol level in cells with overexpressed GCS. These findings suggest that GCS does not generate GlcChol itself, but is required to generate sufficient GlcCer to be used as donor in formation of GlcChol by transglucosylation. This transglucosylation in COS7 cells is particularly mediated by GBA2, and not GBA. The latter notion is consistent with the finding that GBA2 deficiency in mice, and not that of GBA, is accompanied with reduced GlcChol levels of tissues.

GlcChol in Niemann-Pick disease type C mice and U18666A treated cells.

In Niemann Pick disease type C (NPC), cholesterol accumulates prominently in lysosomes as the result of impaired export from the compartment due to defects in either Npcl or Npc 2 (41). In liver of Npcl-deficient mice and the spontaneous spm NPC mice a spectacular, 25-fold, increases in GlcChol content (FIGS. 5A and B) is observed. The identity of the measured glucosylated sterol was examined by digestion with rGBA1. While more than 90% of the GlcChol in liver of normal mice was digested to cholesterol, in the case of material from npc1⁻/npc1⁻ mice this was only 70%. Based on this finding, it seems likely that part of the elevated compound with m/z 572.6>369.4 in NPC liver consists of cholesterol molecules modified differently with sugar, indistinguishable from cholesterol-8-glucoside with the LC-MS method.

To experimentally substantiate the observations made for GlcChol in liver of NPC mice, impaired cholesterol export from lysosomes in RAW 267 cells was induced by exposure to U18666A (42). High intralysosomal cholesterol concentrations appear to favour formation of GlcChol by GBA. This is indicated by the 25-fold elevated GlcChol in liver of two types of NPC mice. In accordance with this, induction of lysosomal cholesterol accumulation with U18666 in cells causes a rapid increase in GlcChol. Concomitant inhibition of lysosomal GBA by conduritol B-epoxide prevented completely the increase of GlcChol in U18666A exposed cells (FIG. 5C). Formation of excessive GlcChol was also prevented by the presence of B-methyl-cyclodextrin, an agent known to reduce intralysosomal cholesterol in NPC cells (43). This indicates that during extreme intralysosomal accumulation of cholesterol, GBA actively generates GIcChol. In normal lysosomes GBA most likely largely degrades the glucosylated sterol,

GlcChol in NPC and GD Patients

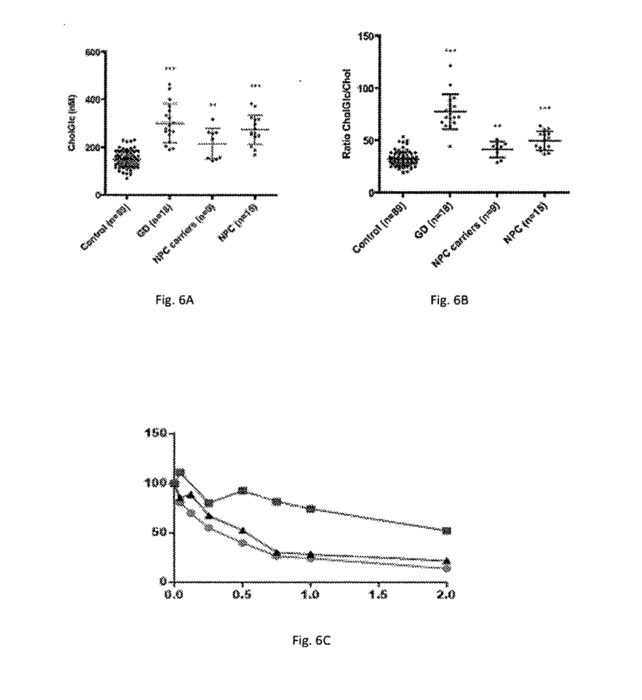

GlcChol levels in plasma of untreated symptomatic type 1 GD patients was determined as well as in NPC patients, carriers and healthy controls. As shown in FIG. 6A, GlcChol tends to be increased in plasma of symptomatic GD patients and less prominently in that of NPC patients. The abnormalities are more pronounced when plasma GlcChol is related to Chol (FIG. 6B). Investigation of plasma specimens of type 1 GD patients treated with Eliglustat, showed a prominent reduction upon inhibition of glycosphingolipid synthesis by the administered GCS inhibitor (FIG. 6C). Matched patients treated with ERT showed a similar response, but not those receiving SRT with Zavesca, a poorer GS inhibitor than Eliglustat (FIG. 6C).

To maximally form GlcChol through transglucosylation high concentrations of GlcCer as donor and high concentrations of cholesterol as acceptor are optimal. Vice versa low high concentrations of ceramide, and low concentrations of GlcCer and cholesterol, will reduce net GlcChol formation. This consideration holds equally for GBA2 and GBA. Fluctuations in sterols and sphingolipids conceivably occur in cells, for example after uptake of cholesterol-rich lipoproteins or upon release of ceramide from sphingomyelin. The ability to maintain some equilibrium between (glucosylated) sphingolipids and sterols by transglucosylating β-glucosidases may have beneficial buffering effects for cells. Of interest in this respect is the present finding that inhibition of GCS leads to reduction of GlcChol in cultured cells, tissues of mice and plasma of GD patients. This strongly suggests that the availability of GlcCer is an important driver of formation of GlcChol through transglucosylation. Since the β-glucosidases GBA and GBA2 also hydrolyse GlcCer, and thus tend to lower its concentration, the exquisite balance of various GlcCer metabolizing enzymes and local cholesterol concentrations will determine Gk.:Choi formation in subcellular compartments.

Materials

25-[N-[(7-nitro-2-1,3-benzoxacliazol-4-yl)methyl]amino]-27-norcholesterol (25-NBD-Cholesterol), N-[6-[(7-nitro-2-1,3-benzoxadiazol-4-yl) amino]hexanoyl]-D-glucosyl-β1-1′-sphingosine (C6-NBD-GlcCer), N-[6-[(7-nitro-2-1,3-benzoxadiazol-4-yl)amino]hexanoyl]-D-erythro-sphingosine (C6-NBD-Cer), D-glucosyl-β-1,1′N-p almitoyl-D-erythro-sphingosine (C16:0-GlcCer), and D-glucosyl-β-1,1′N-oleoyl-D-erythro-sphingosine (C18:1-GIceer) were purchased from Avanti Polar Lipids (Alabaster, Ala., USA). 4-Methylumbelliferyl β-D-glucopyranoside (4MU-Glc) was purchased from Glycosynth™ (Winwick Quay Warrington, Cheshire, England). Conduritol B epoxide (CBE) was from Enzo Life Sciences Inc. (Farmingdale, N.Y., USA), 1-O-cholesteryl-β-D-glucopyranoside (β-cholesteryl glucoside, 8-GlcChol) and ammonium formate (LC-MS quality) were from Sigma-Aldrich (St Louis, Mo., USA). N-(5-adamantane-1-yl-methoxy-pentyl)-deoxynojirimycin (AMP-DNM) and ¹³C6 isotope labelled β-cholesteryl glucoside (¹³C₆-β-GlcChol) were chemically synthesized in the department of Bio-organic Synthesis at the Faculty of Science, Leiden Institute of Chemistry at the University of Leiden (Leiden, The Netherlands). Cerezyme®, a recombinant human GBA1 used in enzyme replacement therapy in Gaucher disease, was obtained from Genzyme (Genzyme Nederland, Naarden, The Netherlands).

LC-MSgrade methanol, 2-propanol, water, HPLC-grade chloroform were purchased from Biosolve; ammonium formate LC-MS grade from Sigma-Aldrich Chemie GmbH.

Mice

The following mice were used for investigation: GD1 mice in which GBA-deficiency was induced (31, 32); mice with spontaneous Niemann Pick Type C (33) and Npc1−/− mice (34); LIMP2−/− mice (35), and GBA2 −/− mice (22). Animals were sacrificed and tissue were immediately frozen and stored at −80° C.

Cloning of cDNAs Encoding GBA2, GBA3 and UCGC

The design of cloning primers was based on NCBI reference sequences NM_172692.3 for murine GBA2, NM_172692.3 for human GBA3 and NM_003358.2 for human UCGC. Using the primers listed below, the full-length coding sequences were cloned into pcDNA3.1/Myc-His (Invitrogen, Life Technologies, Carlsbad, Calif., USA), using primers:

RB143:
GAATTCGCCGCCACCATGGTAACCTGCGTCCCGG
and
RB144:
GCGGCCGCTCTGAATTGAGGTTTGCCAG for mGBA2;
RB252:
GAATTCGCCGCCACCATGGCTTTCCCTGCAGGATTTG
and
RB253:
GCGGCCGCTACAGATGTGCTTCAAGGCC for hGBA3;
RB111:
TCCTGCGGGAGCGTTGTC
and
RB114:
GGTACCTACATCTAGGATTTCCTCTGC for hUCGC.

Cell Culture and Transfection

COS-7 cells were cultured in Iscove's modified Dulbecco's medium (Life Technologies, Carlsbad, Calif., USA) supplemented with 5% fetal bovine serum (FBS; Bodinco, Alkmaar, The Netherlands) and in the presence of penicillin/streptomycin Liffe Technologies, Carlsbad, Calif., USA under 5% CO₂ at 37° C. Cells were seeded at 75% confluence in 6-well plates and transfected using FuGENE® 6 Transfection Reagent (Promega Benelux, Leiden, The Netherlands) according to the manufacturer's instructions, at a FuGENE:DNA ratio of 3:1. After 72 h, the medium was removed, cells were washed trice with ice-cold PBS and harvested by scraping in 25 mM potassium-phosphate buffer, pH 6.5.

In Vitro Assay of Transglucosylase Activity

Homogenates of COS-7 cells overexpressing GBA2, GBA3, GCS, and recombinant GBA1 were used to determine transglucosylase activity of each of the enzymes individually. In principle, the assay was performed as described earlier (11) with a few modifications. First, 40 μL of homogenate of cells overexpressing GBA2, GBA3 or GCS was pre-incubated with 10 μL of 25 mM CBE in water for 20 min on ice (samples containing diluted recombinant GBA1 were pre-incubated in the absence of CBE). To each of the samples 200 μL of the appropriate buffer containing 100 μM of donor (either C18:1-GlcCer or β-CG) and 40 μM of acceptor (either 25-NBD-Cholesterol or C6-NBD-Cer), was added. Transglucosylase activity of GBA2 overexpressing cells was measured in a 150 mM citrate-phosphate buffer, pH 5.8 and the assay for recombinant GBA1 was done in a 150 mM citrate-phosphate buffer, pH 5.2, containing 0.1% BSA, 0.1% Triton-X-100 and 0.2% sodium taurocholate. For GBA3 the assay contained 100 mM Hepes buffer, pH 7.0, The transglycosylase assay for GCS was performed in a 125 mM potassium phosphate buffer pH 7.5 with 12.5 mM UDP-glucose, 6.25 mM MgCl₂, 0.125% BSA, and 0.625% CHAPS. After 1 h of incubation at 37° C., the reaction was terminated by addition of chloroform/methanol (2:1, v/v) and lipids were extracted according to Bligh and Dyer (36). Thereafter lipids were separated by thin layer chromatography on HPTLC silica gel 60 plates (Merck, Darmstadt, Germany) using chloroform/methanol (85:15, v/v) as eluent followed by detection of NBD-labelled lipids using a Typhoon Variable Mode Imager (GE Healthcare Bio-Science Corp., Piscataway, N.J., USA) (37).

Identification of newly formed fluorescent lipid in transglucosylation assays with 25-NBD cholesterol as acceptor as 25-NBD cholesterol was performed following its isolation by scraping from plates by demonstration of complete digestion to NBD-cholesterol using excess recombinant GBA at pH 5.2 (Mcllvaine buffer) in the presence of 0.2% (w/v) sodium taurocholate and 0.1% (v/v) Triton X-100.

Analysis of GlcChol by LC-MS/MS

A Waters Acquity™ TQD instrument was used in all experiments. The instrument consisted of a UPLC system combined with a tandem quadruple mass spectrometer as mass analyser. Data were analysed with Masslynx 4.1 Software (Waters Corporation; Milford Mass.).

GlcChol and ¹³C₆-β-GlcChol (internal standard) were separated using a BEH C18 reversed-phase column (2.1×50 mm, particle size 1.7 μm; Waters), by applying a isocratic elution of mobile phases, 2-propanol:H20 90:10 (v/v) containing 10 mM ammonium formate (Eluent A) and methanol containing 10 mM ammonium formate (Eluent B). The ULPC program was applied during 5.0 minutes consisting of 10% A and 90% B. The divert valve of the mass spectrometer was programmed to discard the UPLC effluent before (0 to 0.25 min) and after (4 to 5 min) the elution of the analytes to prevent system contamination. The flow rate was 0.250 mL/min and the retention time of both GlcChol and the internal standard was 1.43 min (FIG. 2C). The column temperature and the temperature of the auto sampler were kept at 23° C. and 10° C. respectively during the run.

Solutions of GlcChol and ¹³C₆-β-GlcChol and a mixture of both compounds were prepared with concentrations of 1 pmol/μL in 5 mM ammonium formate in methanol. The compounds were introduced in the mass spectrometer by direct infusion and the optimal tuning conditions for both compounds in ES⁺ (electrospray positive) mode were determined (table 1). The most abundant species for both compounds were ammonium adducts, [M+NH₄]⁺, 566.6>369.4 for GlcChol and 572.6>369.4 for ¹³C₆-β-GlcChol (see also FIG. 2B). The product ion represents the cholesterol part of the molecule after loss of the glucose moiety. Because the ¹³C isotopes are on the glucose molecule, the daughter fragment of ¹³C₆-β-GlcCho has the same m/z ratio of 369.4.

TABLE 1
MS/MS instrument parameters

| Capillary voltage | 3.50 | KV |
|---|---|---|
| Cone voltage | 20 | V |
| Source temperature | 120° | C. |
| Desolvation temperature | 450° | C. |
| Cone gas | 50 | L/h |
| Desolvation gas | 950 | L/h |
| Collision gas | 0.20 | mL/min |
| Collision voltage | 20 | V |
| Type | Multiple reaction monitoring | |
| Ion mode | ES⁺ (electrospray positive) | |
| Dwell time | 0.250 | s |
| Interchannel delay | 0.005 | s |
| Interscan delay | 0.005 | s |

| Transitions: | RT(min.): |
|---|---|
| Cholesterol Glucoside | 1.42 |
| ¹³C₆ Cholesterol Glucoside | 1.42 |
| Fit weight | None |
| Smooth method | Mean |
| Smooth width | 2 |

Confirmation of compounds with m/z 566.6>369.4 being GlcChol was performed by demonstration of complete digestion to cholesterol using excess recombinant GBA at pH 5.2 (McIlvaine buffer) in the presence of 0.2% (w/v) sodium taurocholate and 0.1% (v/v) Triton X-100.

Quantification of Total GlcChol in Human Plasma.

For quantitative analysis of GlcChol in samples of plasma, we developed a LC-MS/MS method using the MRM mode of the transitions mentioned above. Firstly, GlcChol was extracted from plasma from a healthy individual according the method of Bligh and Dyer (28) with a few modifications. 20 μL of plasma was pipetted in an Eppendorf tube (2 mL) and 20 μL of an internal standard solution, containing 0.1 pmol/μL of ¹³C₆-β-GlcChol in methanol, was added, followed by addition of 280 μL methanol and 150 μL of chloroform. After brief mixing, the sample was left at room temperature for 30 min, mixed occasionally and centrifuged for 10 min at 15700×g to spin down precipitated protein. The supernatant was transferred to an Eppendorf tube and 150 μL chloroform and 250 μL water were added to induce separation of phases. After centrifugation (5 min at 15700×g) the lower, hydrophobic phase was transferred to a clean Eppendorf tube and the upper phase was washed. by addition of 300 μL of chloroform. Lower phases were pooled and taken to dryness at 35° C. under a nitrogen stream. Next, the residue was dissolved in 700 μL of butanol and 700 μL of water, mixed well and centrifuged for 10 min at 15700 ×g. The upper phase (butanol) was transferred to a 1 mL tube with screw cap and the sample was dried under a gentle stream of nitrogen at 35° C. Subsequently, the residue was dissolved in 150 μL of eluent B by mixing and sonication and after centrifugation (5 min at 15700×g), an aliquot of 100 μL was transferred into an UPLC vial with insert. 10 μL of the solution was injected for analysis.

Secondly, for the quantification of GlcChol in plasma, the sample was spiked with β-GlcChol (concentrations: 0-2.5-5-10-50-100-200-1000 pmol β-GlcChol/mL of plasma), internal standard was added and samples were extracted. The ratio, the area from transition GlcChol over the area from the transition ¹³C₆-β-GlcChol, was plotted against the concentration of GlcChol spiked in the plasma samples. A linear response was obtained over the entire concentration range (y=0.0108×+1.9188, R2=0.998). The within run variation (164.2±4.3 pmol/mL with CV % 2.6) and between run variation (166.8±3.6 pmol/mL with CV % 2.2), was determined in plasma of a healthy volunteer by ten repetitive measurements.

The limit of detection (LOD) was 0.5 pmol/mL plasma with a signal to noise ratio of three and the limit of quantitation (LOQ) was 0.9 pmol/mL plasma with a signal to noise ratio of 10. Calculation of the signal to noise ratio was done using the peak-to-peak method.

Analysis of GlcChol in COS-7 Cells by LC-MS/MS.

COS-7 cells overexpressing GBA2 and/or GCS were homogenized by sonication on ice. Prior to extraction, 2 pmol of ¹³C-labelled GlcChol in methanol (used as an internal standard) was added to 180 μL of homogenate. Next, lipids were extracted according to the method of Bligh and Dyer by addition of methanol, chloroform and water (1:1:0.9, v/v/v) and the lower phase was taken to dryness under a stream of nitrogen. Isolated Is lipids were purified by water/butanol extraction (1:1, v/v) and 8-GlcChol was analyzed by LC-MS as described before.

Analysis of GlcCer and Cer in COS-7 Cells by HPLC

COS-7 cells overexpressing GBA2 and/or GCS, were homogenized by sonication on ice. Prior to extraction, 1 nmol of C17-sphinganine in methanol (used as an internal standard) was added to 100 μL of homogenate. Next, lipids were extracted according to the method of Bligh and Dyer by addition of methanol, chloroform and water (1:1:0.9, v/v/v) and the lower phase was taken to dryness under a stream of nitrogen at 40° C. Isolated lipids were deacylated in a microwave oven, derivatized and analyzed by HPLC as described before (29).

In Vitro Assay of GCase Activity in COS-7 Cells

Homogenates of COS-7 cells overexpressing GBA2 and/or GCS, were analyzed for enzymatic activity of GBA2 using artificial 4MU-Glc substrate as earlier described (38). In short, 12.5 μL of homogenate was incubated with 50 μL of substrate-solution (3 mM 4MU-Glc in 150 mM citrate-phosphate buffer, pH 5.8) for 20 or 120 min at 37° C. The reaction was terminated by addition of 1.25 mL stop-buffer (0.3 M Glycine/NaOH, pH 10.6) and the released 4MU was measured with a LS55 Fluorescence Spectrometer (Perkin-Elmer, Waltham, Mass., USA).

SUPPLEMENTAL TABLE 1
Table 1. Degradation of GlcChol by GBA and GBA2.

| | Input: nmol4MU-β-Glc | Percentage digestion GlcChol |
|---|---|---|
| | hydrolysis per ml/h | after 1 h (10 nmole) |
| rGBA1 | 15 | 95% |
| GBA2 | 18 | 83% |

### REFERENCES

1. Wennekes T, van den Berg R J B F I N, Boot R G, van der Marel G A, Overkleeft H S, Aerts J M F G. Glycosphingolipids-Nature, Function and Pharmacological Modulation. Angew Chem Int Ed Engl. 2009; 48(47):8848-69.
2. Pata M O, Hannun Y A, Ng C K. Plant sphingolipids: decoding the enigma of the Sphinx. New Phytol. 2010 February; 185(3):611-30.
3. Wu W, Narasaki R, Maeda F, Hasumi K. Glucosyldiacylglycerol enhances reciprocal activation of prourokinase and plasminogen. Biosci Biotechnol Biochem. 2004 July; 68(7):1549-56.4. Watanabe T, Ito T, Coda H M, Ishibashi Y, Miyamoto T, Ikeda K, Taguchi R, Okino N, Ito M. Sterylglucoside catabolism in Cryptococcus neoformans with endoglycoceramidase-related protein 2 (EGCrP2), the first steryl-8-glucosidase identified in fungi. J Biol Chem. 2015 Jan 9;290(2):1005-19.
5. Kunimoto S, Kobayashi T, Kobayashi S, Murakami-Murofushi K. Expression of cholesteryl glucoside by heat shock in human fibroblasts. Cell. Stress Chaperones. 2000 January; 5(1):3-7.
6. Kunimoto S, Murofush.i W, Yamatsu I, Hasegawa Y, Sasaki N, Kobayashi S, Kobayashi T, Murofushi H, Murakami-Murofushi K. Cholesteryl glucoside-induced protection against gastric ulcer. Cell Struct Funct. 2003 June; 28(3):179-86.
7. Kunimoto S, Murofushi W, Kai FI, Ishida Y, Uchiyama A, Kobayashi T, Kobayashi S, Murofushi H, Murakami-Murofushi K. Steryl glucoside is a lipid mediator in stress-responsive signal transduction. Cell Struct Funct. 2002 June; 27(3):157-62.
8. Ichikawa S, Sakiyama H, Suzuki G, Hidari K I, Hirabayashi Y. Expression cloning of a cDNA for human ceramide glucosyltransferase that catalyzes the first glycosylation step of glycosphingolipid synthesis. Proc Natl Acad Sci USA. 1996 Oct. 29; 93(22):12654.
9. van Meer G, Wolthoorn J, Degroote S. The fate and function of glycosphingolipid glucosylceramide. Philos Trans R Soc Lond B Biol Sci. 2003 May 29; 358(1433):869-73.
10. Akiyama H, Sasaki N, Hanazawa S, Gotoh M, Kobayashi S, Hirabayashi Y, Murakami-Murofushi K. Novel sterol glucosyltransferase in the animal tissue and cultured cells: evidence that glucosylceramide as glucose donor. Biochim Biophys Acta. 2011 May; 1811(5):314-22.
11. Akiyama H, Kobayashi S, Hirabayashi Y, Murakami-Murofushi K. Cholesterol glucosylation is catalyzed by transglucosylation reaction of β-glucosidase 1. Biochem Biophys Res Commun. 2013 Nov. 29; 441(4):838-43.
12. Vanderjagt D J, Fry D E, Glew R H. Human glucocerebrosidase catalyses transglucosylation between glucocerebroside and retinol. Biochem J. 1994 Jun. 1; 300 (Pt 2):309-15.
13. Beutler E, Grabowski G A. Gaucher disease. In: Striver C R, Beadet A L, Sly W S, Valle D, eds. The Metabolic and Molecular Bases of Inherited Disease. 7th ed. New York, N.Y.: McGraw-Hill; 1995: 2641-2670.
14. Ferraz M J, Kallemeijn W W, Mirzaian M, Herrera Moro D, Marques A, Wisse P, Boot R G, Willems L I, Overkleeft H S, Aerts J M. Gaucher disease and Fabry disease: new markers and insights in pathophysiology for two distinct glycosphingolipidoses. Biochim Biophys Acta. 2014 May; 1841(5):811-25.
15. Dekker N, van Dussen L, Hollak C E, Overkleeft H, Scheij S, Ghauharali K, van Breemen M J, Ferraz M J, Groener J E, Maas M, Wijburg F A, Speijer D, Tylki-Szymanska A, Mistry P K, Boot R G, Aerts J M. Elevated plasma glucosylsphingosine in Gaucher disease: relation to phenotype, storage cell markers, and therapeutic response. Blood. 2011 Oct. 20; 118(16):e 118-27.
16. Rolfs A, Giese A K, Grittner U, Mascher D, Elstein D, Zimran A, Bottcher T, Lukas J, Hubner R, Golnitz U, Rohle A, Dudesek A, Meyer W, Wittstock M, Mascher H. Glucosylsphingosine is a highly sensitive and specific biomarker for primary diagnostic and follow-up monitoring in Gaucher disease in a non-Jewish, Caucasian cohort of Gaucher disease patients. PLoS One. 2013 Nov. 20; 8(11):e79732.
17. Barton N W, Brady R O, Dambrosia J M, Di Bisceglie A M, Doppelt S H, Hill S C, Mankin H J, Murray G J, Parker R I, Argoff C E, et al. Replacement therapy for inherited enzyme deficiency—macrophage-targeted glucocerebrosidase for Gaucher's disease. N Engl J Med. 1991 May 23; 324(21):1464-70.
18. Cox T, Lachmann R, Hollak C, Aerts J, van Weely S, Hrebicek M, Platt F, Butters T, Dwek R, Moyses C, Gow I, Elstein D, Zimran A. Novel oral treatment of Gaucher's disease with N-butyldeoxynojirimycin (OGT 918) to decrease substrate biosynthesis. Lancet. 2000 Apr. 29; 355(9214):1481-5.
19. Cox T M, Drelichman G, Cravo R, Balwani M, Burrow T A, Martins A M, Lukina E, Rosenbloom B, Ross L, Angell J, Puga A C. Eliglustat compared with imiglucerase in patients with Gaucher's disease type 1 stabilised on enzyme replacement therapy: a phase 3, randomised, open-label, non-inferiority trial. Lancet. 2015 Mar. 25. pii: S0140-6736(14)61841-9.
20. Hughes D A, Pastores G M. Eliglustat for. Gaucher's disease: trippingly on the tongue. Lancet. 2015 Mar. 25. pii: S0140-6736(15)60206-9.
21. van Weely S, Brandsma M, Strijland A, Tager J M, Aerts J M. Demonstration of the existence of a second, non-lysosomal glucocerebrosidase that is not deficient in Gaucher disease. Biochim Biophys Acta. 1993 Mar. 24; 1181(1):55-62.
22. Yildiz Y, Matern H, Thompson B, Allegood J C, Warren R L, Ramirez D M, Hammer R E, Hamra F K, Matern S, Russell D W. Mutation of beta-glucosidase 2 causes glycolipid storage disease and impaired male fertility. J Clin Invest. 2006 November; 116(11):2985-94.
23. Boot R G, Verhoek M, Donker-Koopman W, Strijland A, van Marle J, Overkleeft I I S, Wennekes T, Aerts J M. Identification of the non-lysosomal glucosylceramidase as beta-glucosidase 2. J Biol Chem. 2007 Jan. 12; 282(2):1305-12.
24. Burke D G, Rahim A A, Waddington S N, Karlsson S, Enquist I, Bhatia K, Mehta A, Vellodi A, Heales S. Increased glucocerebrosidase (GBA) 2 activity in GBA1 deficient mice brains and in Gaucher leucocytes. J Inherit Metab Dis. 2013 September; 36(5):869-72.
25. Dekker N, Voorn-Brouwer T, Verhoek M, Wennekes T, Narayan R S, Speijer D, Hollak C E, Overkleeft H S, Boot R G, Aerts J M. The cytosolic β-glucosidase GBA3 does not influence type 1 Gaucher disease manifestation. Blood Cells Mol Dis. 2011 Jan. 15; 46(1): 19-26.
26. Kittl R, Withers S G. New approaches to enzymatic glycoside synthesis through directed evolution. Carbohydr Res. 2010 Jul. 2; 345(10):1272-9.
27. Ghisaidoobe A T, van den Berg R J, Butt S S, Strijland A, Donker-Koopman W E, Scheij S, van den Nieuwendijk A M, Koomen G J, van Loevezijn A, Leemhuis M, Wennekes T, van der Stelt M, van der Marel G A, van Boeckel C A, Aerts J M, Overkleeft H S. Identification and development of biphenyl substituted iminosugars as improved dual glucosylceramide synthase/neutral glucosylceramidase inhibitors. J Med Chem. 2014 Nov. 13; 57(21):9096-104.
28. Overkleeft H S, Renkema G H, Neele J, Vianello P, Hung I O, Strijland A, van der Burg A M, Koomen G J, Pandit U K, Aerts J M. Generation of specific deoxynojirimycin-type inhibitors of the non-lysosomal glucosylceramidase. J Biol Chem. 1998 Oct. 9; 273(41):26522-7
29. Witte M D, Kallemeijn W W, Aten J, Li K Y, Strijland A, Donker-Koopman W E, van den Nieuwendijk A M, Bleijlevens B, Kramer G, Florea B I, Hooibrink B, Hollak C E, Ottenhoff R, Boot R G, van der Marel G A, Overkleeft H S, Aerts J M. Ultrasensitive in situ visualization of active glucocerebrosidase molecules. Nat Chem Biol. 2010 Dec;6(12):907-13.
30. Witte M D, Walvoort M T, Li K Y, Kallemeijn W W, Donker-Koopman W E, Boot R G, Aerts J M, Codee J D, van der Marel G A, Overkleeft H S. Activity-Based Profiling of Retaining β-Glucosidases: A Comparative Study. Chembiochem. 2011 May 16; 12(8): 1263-9.
31. Enquist I B, Nilsson E, Ooka A, Mansson J E, Olsson K, Ehinger M, Brady R O, Richter J, Karlsson S. Effective cell and gene therapy in a murine model of Gaucher disease. Proc Natl Acad Sci U S A. 2006 Sep. 12; 103(37):13819-24.
32. Dahl M, Doyle A, Olsson K, Mansson J E, Marques A R, Mirzaian M, Aerts J M, Ehinger M, Rothe M, Modlich U, Schambach A, Karlsson S. Lentiviral gene therapy using cellular promoters cures type 1 Gaucher disease in mice. Mol Ther. 2015 May; 23(5):835-44.
33. Pentchev P G, Boothe A D, Kruth H S, Weintroub H, Stivers J, Brady R O. A genetic nstorage disorder in BALB/C mice with a metabolic block in esterification of exogenous cholesterol. J Biol Chem. 1984 May 10; 259(9):5784-91.
34. Loftus S K, Morris J A, Carstea E D, Gu J Z, Cummings C, Brown A, Ellison J, Ohno K, Rosenfeld M A, Tagle D A, Pentchev P G, Pavan W J. Murine model of Niemann-Pick C disease: mutation in a cholesterol homeostasis gene. Science. 1997 Jul. 11; 277(5323):232-5Loftos mice
35. Gamp A C, Tanaka Y, Lullmann-Rauch R, Wittke D, D'Hooge R, De Deyn P P, Moser T, Maier H, Hartmann D, Reiss K, Illert A L, von Figura K, Saftig P. LIMP-2/LGP85 deficiency causes ureteric pelvic junction obstruction, deafness and peripheral neuropathy in mice. Hum Mol Genet. 2003 Mar. 15; 12(6):631-46.
36. Bligh E G, Dyer W J. A rapid method of total lipid extraction and purification. Can J Biochem Physiol. 1959 August; 37(8):911-7.
37. Van Weely S, Van Leeuwen M B, Jansen I D, De Bruijn M A, Brouwer-Kelder E M, Schram A W, Sa Miranda M C, Barranger J A, Petersen E M, Goldblatt J, et al. Clinical phenotype of Gaucher disease in relation to properties of mutant glucocerebrosidase in cultured fibroblasts. Biochim Biophys Acta. 1991 Jun. 5; 1096(4):301-11.
38. Kallemeijn W W, Li K Y, Witte M D, Marques A R, Aten J, Scheij S, Jiang J, Willems L I, Voorn-Brouwer T M, van Roomen C P, Ottenhoff R, Boot R G, van den Elst H, Walvoort M T, Florea B I, Codee J D, van der Marel G A, Aerts J M, Overkleeft H S. Novel activity-based probes for broad-spectrum profiling of retaining β-exoglucosidases in situ and in vivo. Angew Chem Int Ed Engl. 2012 Dec. 7; 51(50):12529-33.
39. Reczek D, Schwake M, Schroder J, Hughes H, Blanz J, Jin X, Brondyk W, Van Patten S, Edmunds T, Saftig P. LIMP-2 is a receptor for lysosomal mannose-6-phosphate-independent targeting of beta-glucocerebrosidase. Cell. 2007 Nov. 16; 131(4):770-83.
40. Gaspar P, Kallemeijn W W, Strijland A, Scheij S, Van Eijk M, Aten J, Overkleeft H S, Balreira A, Zunke F, Schwake M, Sd Miranda C, Aerts J M. Action myoclonus-renal failure syndrome: diagnostic applications of activity-based probes and lipid analysis. J Lipid Res. 2014 January; 55(1):138-45.Gaspar J L R 41 Tangirala R K, Mahlberg F H, Glick J M, Jerome W G, Rothblat G H. Lysosomal accumulation of unesterified cholesterol in model macrophage foam cells. J Biol. Chem. 1993 May 5; 268(13):9653 60.
42. Liscum L. Pharmacological inhibition of the intracellular transport of low-density lipoprotein-derived cholesterol in Chinese hamster ovary cells. Biochim Biophys Acta. 1990 Jun. 28; 1045(1):40-8.
43. Vance J E, Karten B. Niemann-Pick C disease and mobilization of lysosomal cholesterol by cyclodextrin. J Lipid Res. 2014 Mar 24; 55(8):1609-1621.
44. Nair 8, Boddupalli C S, Verma R, Liu J, Yang R, Pastores G M, Mistry P K, Dhodapkar M V. Type II NKT-TFH cells against Gaucher lipids regulate B-cell immunity and inflammation. Blood. 2015 Feb. 19; 125(8):1256-71.
45. Grille S, Zaslawski A, Thiele S, Plat J, Warnecke D. The functions of steryl glycosides come to those who wait: Recent advances in plants, fungi, bacteria and animals. Prog Lipid Res. 2010 Jul; 49(3):262-88.
46. Neculai D, Schwake M, Ravichandran M, Zunke F, Collins R F, Peters J, Neculai M, Plumb J, Loppnau P, Pizarro J C, Seitova A, Trimble W S, Saftig P, Grinstein S, Dhe-Paganon S. Structure of LIMP-2 provides functional insights with implications for SR-BI and CD36. Nature, 2013 Dec. 5; 504(7478):172-6.

1. A method of detecting a glycosylated metabolite in a sample comprising:
adding to the sample a compound comprising a labelled glycosyl group coupled via an O-glycosidic bond to an aglycon and of the following structural formula: 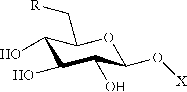
wherein
R=a hydroxyl or a detection label; and
X=an aglycon;
wherein the glycosyl group comprises at least one isotope and/or an R side chain being a label for detection, and
wherein the sample is screened for the presence of a glycosylated metabolite with the glycosyl group other than a glucosylceramide.

2. The method according to claim 1, wherein the glycosylated metabolite is a glucosylated or xylosylated metabolite, in particular a glucosylated or xylosylated sterol, (diacyl)glycerol, retinol, tocopherol, geraniol, farnesol, serine, threonine, or tryptophan.

3. The method according to claim 1, wherein the glycosyl group comprises at least one ¹³C-isotope for detection using LC-MS/MS.

4. The method according to claim 1, wherein X is selected from a group consisting of 4-methylumbelliferone, p-nitrophenol, dopamin, serotonin, vitamin D precursor, calciferol or cholecalciferol, dihydrocalciferol, monoacylglycerol (endocannabinoid), (diacyl)glycerol, retinol, tocopherol, geraniol, farnesol, serine, threonine, tryptophan, and sterol, in particular ceramide or cholesterol.

5. The method according to claim 1, wherein the sample comprises a glycosyltransferase, in particular a glucocerebrosidase or a glucosylceramidase.

6. The method according to claim 1, wherein the sample is a sample from a subject suffering from a disorder caused by accumulation of excessive amounts of a glycosylated metabolite, preferably other than glucosylceramide.

7. The method according to claim 6, wherein the disorder is one or more of lysosomal glycosphingolipid storage disorder, osteoporosis, abnormal vitamin D metabolism or an alpha-synucleinopathy (Parkinson's, Lew-Body dementia).

8. A method of treating a disorder caused by accumulation of a glycosylated metabolite other than glucosylceramide in a subject:
administering to a subject in need thereof an effective amount of a glucosvIceramide synthase (GCS) inhibitor.

9. The method of claim 8, wherein the GCS inhibitor is selected from the group consisting of miglitol, miglustat, eliglustat or a biphenyl-substituted deoxynojirimycin derivative.

10. The method of claim 8, wherein the biphenyl-substituted deoxynojirimycin derivative is a biphenyl-substituted D-gluco-deoxynojirimycin or a biphenyl-substituted L-ido-deoxynojirimycin, in particular a biphenyl-substituted L-ido-deoxynojirimycin selected from the group with the following structural formulas: 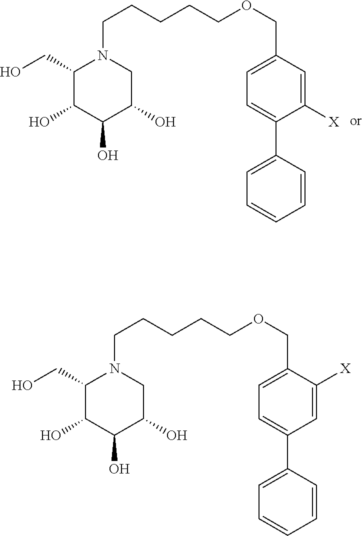
wherein X is F or CF₃.

11. The method of claim 8, wherein the glycosylated metabolite is a dopamine, serotonin, vitamin D precursor, calciferol, cholecalciferol, (diacyl)glycerol, retinol, tocopherol, geraniol, farnesol, serine, threonine, tryptophan or sterol, in particular cholesterol.

12. The method of claim 8, wherein the disorder is one or more of osteoporosis, abnormal vitamin D metabolism or an alpha-synucleinopathy (Parkinson's, Lew-Body dementia).

13. A method of typing a sample from an individual, comprising:
determining a level of at least one glycosylated metabolite other than glucosylceramide in a relevant sample from the individual,
comparing said level with a reference; and
typing said sample as a sample from an individual with a predisposition for one or more of the disorders lysosomal glycosphingolipid storage disorder, osteoporosis, abnormal vitamin D metabolism or an alpha-synucleinopathy on the basis of said comparison.

14. The method of typing a sample according to claim 13, wherein the at least one glycosylated metabolite is one or more of group comprising sterol, (diacyl)glycerol, retinol, tocopherol, geraniol, farnesol, serine, threonine, or tryptophan.

15. (canceled)


SEQUENCE LISTING (6 sequences)

SEQ ID NO: 1 - DNA, 34 bp, Artificial Sequence
  primer
gaattcgccgccaccatggtaacctgcgtcccgg

SEQ ID NO: 2 - DNA, 28 bp, Artificial Sequence
  primer
gcggccgctctgaattgaggtttgccag

SEQ ID NO: 3 - DNA, 37 bp, Artificial Sequence
  primer
gaattcgccgccaccatggctttccctgcaggatttg

SEQ ID NO: 4 - DNA, 28 bp, Artificial Sequence
  primer
gcggccgctacagatgtgcttcaaggcc

SEQ ID NO: 5 - DNA, 18 bp, Artificial Sequence
  primer
tcctgcgggagcgttgtc

SEQ ID NO: 6 - DNA, 27 bp, Artificial Sequence
  primer
ggtacctacatctaggatttcctctgc